(19) United States
(12) Patent Application Publication
(10) Pub. No.: US 2005/0065613 A1
(43) Pub. Date: Mar. 24, 2005

(54) Reinforced fusion implant

(76) Inventors: Jeffrey M. Gross, Memphis, TN (US); Thomas A. Simonton, Memphis, TN (US)

Correspondence Address: Woodard, Emhardt, Moriarty, McNett & Henry LLP;Bank One Center/Tower, Suite 3700, 111 Monument Circle, Indianapolis, IN, 46204-5137, US

(21) Appl. No.: 10/669,764

(22) Filed: Sep. 24, 2003

(51) Int. Cl. A61F 2/28; A61F 2/44

(52) U.S. Cl. 623/23.51; 623/17.11

(57) ABSTRACT

The present invention provides a fusion implant for use between adjacent bony structures and related methods. The implant includes a body and at least one reinforcing member positionable within the body.

### BACKGROUND

Implants for use in fusing adjacent bony structures facilitate fusion by maintaining the adjacent bony structures in a predetermined spaced relationship while bone grows between them. In some cases these implants are formed from body tissues. In forming a fusion implant from body tissue, a source of tissue, such as a bone, is formed into pieces meeting the desired shape and strength requirements for a particular implant. In the case of bone, the requirements are often specified in terms of a minimum wall thickness, minimum load bearing capacity, and/or geometric size and shape. A portion of the source tissue, including pieces removed in forming implants, will fall short of the requirements to form an integral implant. Thus, it is often difficult to obtain a high yield from a particular source. In other cases, it is desirable to utilize a fusion implant comprising relatively less dense material, such as for example cancellous bone, to promote bone growth between the adjacent bony structures. However, such material may need reinforcement to enable it to support the required load.

### SUMMARY

The present invention provides a fusion implant for use between adjacent bony structures, for example, such as to facilitate fusion of the bony structures.

In one aspect of the invention, a fusion implant for insertion between adjacent bony structures comprises a body having opposing sides for contacting the adjacent bony structures; and at least one member positioned in the body, the member having a first end and a second end, the member having a tapered portion between the first and second ends.

In another aspect of the invention, a fusion implant for insertion between adjacent bony structures in load bearing arrangement comprises a body comprising bone and having opposing sides for contacting the adjacent bony structures; and a structural member comprising bone positioned in the body such that the load carrying capacity of the implant is increased, the member having a first end and a second end, the member having a tapered portion between the first and second ends.

In another aspect of the invention, a fusion implant for insertion between adjacent bony structures in load bearing arrangement comprises a body having opposing sides for contacting the adjacent bony structures; and a structural member positioned in the body such that the load carrying capacity of the implant is increased, the member having a first end and a second end, the member extending only partway through the body.

In another aspect of the invention, a fusion implant for insertion between adjacent bony structures in load bearing arrangement comprises a body having first and second opposing sides for contacting the adjacent bony structures; and a member positioned in the body, the member having a first end adjacent the first opposing side and a second end spaced toward the second opposing side, an enlarged head being formed adjacent the first end and a shaft extending from the head toward the second end.

In another aspect of the invention, a fusion implant for insertion between adjacent bony structures in load bearing arrangement comprises a body having first and second opposing sides for contacting the adjacent bony structures; and at least one member positioned in the body and extending from each of the first and second opposing sides partway toward the other opposing side, a portion of the at least one member extending from each side overlying a portion of the at least one member extending from the opposite side, the overlying portions being spaced from one another such that a predetermined amount of load induced subsidence of the members is permitted relative to each other within the body.

In another aspect of the invention, a fusion implant for insertion between adjacent bony structures in load bearing arrangement comprises a body having first and second opposing sides for contacting the adjacent bony structures; and a member positionable to extend within the body from at least one of the first and second opposing sides, the member having a first surface that receives a load from one of the bony structures and a second surface, oblique to the first surface, that transmits the load to the body.

In another aspect of the invention, a fusion implant for insertion between adjacent bony structures in load bearing arrangement comprises a body having first and second opposing sides for contacting the adjacent bony structures; and a member positionable to extend within the body from at least one of the first and second opposing sides, the member having a first portion with a first cross-sectional area that receives a load from one of the bony structures and a second portion with a second cross-sectional area that transmits the load to the body.

In another aspect of the invention, a fusion implant for insertion between adjacent bony structures in load bearing arrangement comprises a body having first and second opposing sides for contacting the adjacent bony structures; a first member positionable to extend within the body from one of the first and second opposing sides, the first member comprising a first body having a first end and a second end, wherein the first end is positionable adjacent one of the opposing bony structures, and wherein the first body has a tapered portion between the first and second ends; a second member positionable to extend within the body from one of the first and second opposing sides, the second member comprising a second body having a third end and a fourth end, wherein the third end is positionable adjacent one of the opposing bony structures, and wherein the second body has a tapered portion between the first and second ends; and wherein at least a portion of the second member and at least a portion of the first member each lie along a line substantially corresponding to a load bearing axis between the opposing bony structures.

In another aspect of the invention, a fusion implant for insertion between adjacent bony structures in load bearing arrangement comprises a body having first and second opposing sides for contacting the adjacent bony structures; a first member positionable to extend within the body from one of the first and second opposing sides; a second member positionable to extend within the body from one of the first and second opposing sides opposite the first member; and wherein at least a portion of the first member and at least a portion of the second member each lie along a line substantially corresponding to a load bearing axis between the opposing bony structures, the body having a first area that receives load from the first member and a second area that transmits load to the second member.

In another aspect of the invention, a method of treating adjacent bony structures comprises providing a fusion implant having a body having opposing sides for contacting the adjacent bony structures and a member positioned in the body, the member having a first end and a second end, the member having a tapered portion between the first and second ends; and positioning the implant between the adjacent bony structures in load bearing arrangement.

In another aspect of the invention, a method of treating adjacent bony structures comprises providing a fusion implant having a body having opposing sides for contacting the adjacent bony structures and a member positioned in the body, the member having a first end and a second end, the member extending only partway through the body; and positioning the implant between the adjacent bony structures in load bearing arrangement.

In another aspect of the invention, a method of treating adjacent bony structures comprises providing a fusion implant having a body having first and second opposing sides for contacting the adjacent bony structures and at least one member positioned in the body and extending from each of the first and second opposing sides partway toward the other opposing side, a portion of the at least one member extending from each side overlying a portion of the at least one member extending from the opposite side, the overlying portions being spaced from one another such that a predetermined amount of load induced subsidence of the members is permitted relative to each other within the body; and positioning the implant between the adjacent bony structures in load bearing arrangement.

In another aspect of the invention, a method of making a fusion implant for insertion between adjacent bony structures in load bearing arrangement comprises forming a body; and positioning a member in the body, the member having a first end and a second end, the member having a tapered portion between the first and second ends.

In another aspect of the invention, a method of making a fusion implant for insertion between adjacent bony structures in load bearing arrangement comprises forming a body having opposing sides for contacting the adjacent bony structures; and positioning a member in the body, the member having a first end and a second end, the member extending only partway through the body.

In another aspect of the invention, a method of making a fusion implant for insertion between adjacent bony structures in load bearing arrangement comprises forming a body having first and second opposing sides for contacting the adjacent bony structures; and positioning at least one member in the body extending from each of the first and second opposing sides partway toward the other opposing side, a portion of the at least one member extending from each side overlying a portion of the at least one member extending from the opposite side, the overlying portions being spaced from one another such that a predetermined amount of load induced subsidence of the members is permitted relative to each other within the body.

In another aspect of the invention, a system for use in fusing adjacent bony structures, comprises a body having first and second opposing sides for contacting the adjacent bony structures; a member positionable to extend within the body from at least one of the first and second opposing sides, the member having a first surface that receives a load from one of the bony structures and a second surface, oblique to the first surface, that transmits the load to the body; and a fixation device attachable to the adjacent bony structures and having a structure to limit relative motion between the adjacent bony structures.

In another aspect of the invention, a system for use in fusing adjacent bony structures, comprises a body having first and second opposing sides for contacting the adjacent bony structures; a member positionable to extend within the body from at least one of the first and second opposing sides, the member having a first portion with a first cross sectional area that receives a load from one of the bony structures and a second portion with a second cross sectional area that transmits the load to the body; and a fixation device attachable to the adjacent bony structures and having a structure to limit relative motion between the adjacent bony structures.

### BRIEF DESCRIPTION OF THE DRAWINGS

Various embodiments of the present invention will be discussed with reference to the appended drawings. These drawings depict only illustrative embodiments of the invention and are not to be considered limiting of its scope.

FIG. 1 is a perspective view of an illustrative implant according to the present invention.

FIG. 2 is a sectional view taken along line 2-2 of FIG. 1.

FIG. 3 is a sectional view like FIG. 2 showing an optional arrangement of members within the implant.

FIG. 4 is a side elevation view of an optional configuration of a member for inclusion in the implant of FIG. 1.

FIG. 5 is a bottom plan view of the configuration of FIG. 4.

FIG. 6 is a top plan view of an optional configuration of a member for inclusion in the implant of FIG. 1.

FIG. 7 is a perspective view of a fusion implant like that of FIG. 1 shown with an optional member.

FIG. 8 is a sectional view of a fusion implant like that of FIG. 1 shown with an optional member.

FIG. 9 is a side elevation view of the implant of FIG. 1 positioned between adjacent bony structures and shown with an optional fixation device.

### DETAILED DESCRIPTION

Embodiments of a fusion implant include a body for placing between adjacent bony structures and one or more reinforcing members positioned in the body. The combination may form a load bearing implant. A reinforcing member may have a load bearing capacity greater than the load bearing capacity of the body such that the load carrying capacity of the implant is increased. The adjacent bony structures may include vertebrae, long bones, and cranial bones, among others.

The body has surfaces for contacting the adjacent bony structures and may be shaped to fill some or all of the space between the adjacent bony structures to maintain the bony structures in a desired spaced relationship during healing or fusion. The body may provide structural support up to the limits of its load bearing capacity. The body may include cancellous bone, cortical bone, uni-cortical bone, bi-cortical bone, tri-cortical bone, demineralized bone, partially demineralized bone, metal, polymer, resorbable polymer, ceramic, bioglass, and/or other suitable materials. For example the body may comprise a block of cancellous bone, with or without one or more cortical faces adjacent the cancellous bone.

In the case of the body or member comprising bone, the bone may be obtained from any suitable bone source including the implant recipient as in an autograft, another source of the same species as in an allograft, or a source of a different species as in a xenograft. Suitable examples of musculoskeletal tissue include ilium, humerus, tibia, femur, fibula, patella, ulna, radius, rib, vertebral bodies, and/or other suitable bones. The bone pieces may be machined, cut, planed, and/or otherwise removed and/or formed from the donor bone.

In the case where the body or member includes polymers, the polymers may include polyethylene, polyester, polyglycolic acid, polylactic acid, polyaryletherketone, polyetheretherketone, polytetrafluroethylene, and/or other suitable polymers and combinations thereof.

The body may include one or more openings to facilitate fusion of the adjacent bony structures. The body may include a material to promote fusion of the adjacent bony structures incorporated into the body itself or placed in openings formed in the body. Such bone growth-promoting material may include bone paste, cancellous bone, bone chips, bone morphogenic protein (BMP), LIM mineralization protein (LMP), platelet derived growth factors, bone marrow aspirate, stem cells, biologic growth factors, and/or other suitable materials and combinations thereof.

The one or more members positioned within the body may add load bearing capacity and/or axial stiffness to the implant. The member may permit a predetermined amount of size reduction of the implant in response to load. For example, opposing members may be spaced so as to allow a predetermined subsidence of the members into the body under load. The member may include any form and any biocompatible material capable of withstanding a predetermined load.

The member may have a variety of shapes to facilitate transmitting load to or receiving load from the body. For example, the member may have a first surface that receives a load from one of the bony structures and a second surface that transmits the load to the body. The second surface may be oblique to the first surface. The member may have a tapered portion between the first and second ends. A tapered portion may increase the contact area between the body and member as compared to a non-tapered configuration. It may also provide a positive engagement between the body and implant to resist pushing of the member from the body. The member may be positioned with a tapered portion that tapers from a first bone contacting side of the body toward a second bone contacting side such that the member has a larger end that is exposed and a smaller end that is contained within the body.

Alternatively, the member may have a first portion with a first cross-sectional area that receives a load from one of the bony structures and a second portion with a second cross-sectional area that transmits the load to the body. The first cross-sectional area may be larger than the second cross sectional area. Furthermore, the first portion may be adjacent one of the bone contacting sides of the implant and the second portion may be spaced from the first portion into the body.

The member may have an enlarged head and a shaft extending from the head to provide positive engagement of the member in the body. The head and/or shaft may also include a tapered portion.

The member may be in the form of particles, strips or sticks, blocks, or beams. For example, a beam may have a cross sectional shape that is round, rectangular, “I”-shaped, “T”-shaped, “C”-shaped or other suitable shape. It may be cylindrical, rectangular, tapered, hour glass shaped, or other suitable longitudinal shape. The member may be made from bone, metal, ceramic, carbon, bioglass, and/or polymers and combinations thereof. If it is of bone, each piece of bone may comprise cortical bone, uni-cortical bone, bi-cortical bone, and/or tri-cortical bone for achieving a predetermined load-bearing capability in the implant. For example, the member may comprise a piece of cortical bone positioned within the body. Additionally, each piece or strip of bone may comprise cancellous bone. Further, the pieces of bone may be mineralized, partially demineralized, fully demineralized, or combinations thereof. If the structural member includes polymers, they may be resorbable or non-resorbable and include polyethylene, polyester, polyglycolic acid, polylactic acid, polyaryletherketone, polyetheretherketone, polytetrafluroethylene, and/or other suitable polymers and combinations thereof.

The member and body may comprise materials having different mechanical properties. The portion of the body surrounding the member may be relatively softer than the member such that the portion surrounding the member is deformed to fit closely around the member. The member may be stiffer than the body or have a higher load carrying capacity than the body. For example, the portion of the body surrounding the member may comprise cancellous bone and the member may comprise cortical bone.

The member may include any form and any biocompatible material capable of withstanding a predetermined load. Combining the body and member into an implant allows the use of body materials having less than a predetermined minimum load bearing capacity and/or a predetermined geometry outside of a predetermined standard. The combination forms an assembled load-bearing implant that achieves the predetermined capacity and/or geometry.

The member may extend through the body, may extend from one side partway through the body such that the member is exposed at a first opposing side and stops short of a second opposing side, or it may be embedded within the body, such that it is surrounded on all sides by the body.

The implant may be used in conjunction with a fixation device to form a bone fixation system. The fixation device may be attached to the adjacent bony structures to limit the relative motion between them. The fixation device may substantially prevent all relative motion, or it may allow a predetermined amount of motion during the healing and fusion processes.

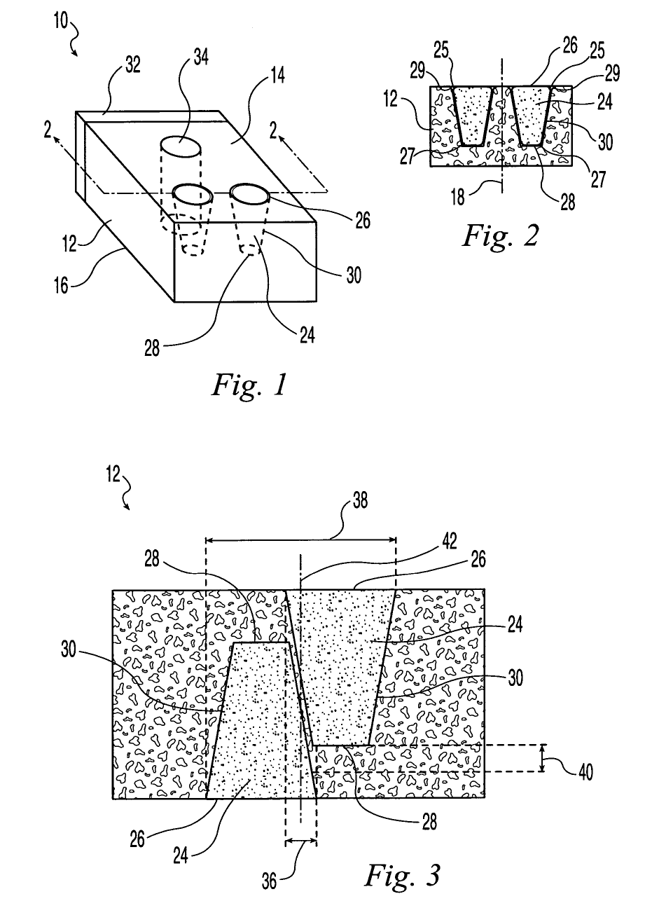

Referring to FIGS. 1 and 2, an illustrative embodiment of a fusion implant 10 includes a body 12 having first 14 and second 16 opposing sides for implantation between adjacent bony structures. A load bearing axis 18 extends between the first 14 and second 16 sides. A member 24 is positioned in the body 12. The member 24 reinforces the implant 10 by increasing its load bearing capacity and/or stiffness. The member includes a first end 26, a second end 28, and a tapered portion 30 between the first 26 and second 28 ends. In this example, the member comprises a truncated conical solid tapering from a larger first end 26 to a smaller second end 28 in a direction parallel to the load bearing axis 18. The first end 26 is positioned adjacent the first opposing side 14 of the body 12 so that it is exposed and may contact an adjacent bony structure. The second end 28 is spaced toward the second opposing side 16 but stops short such that it is contained or hidden within the body. In this configuration, a first area receives a load which is transmitted via a second area to the body. For example, the first end 26 may receive load from adjacent bony structures and the second end 28 and side wall of the tapered portion 30 distribute the load to the body 12. With the second end 28 contained within the body 12, all of the load received by the member 24 is transmitted to the body 12. Where the member is stiffer than the body, this load transmission results in an implant 10 that is less stiff than if the member 24 extended through the body 12. The body 12 acts as a load intermediate. It also allows some load induced compression of the body. Where bone growth is expected to occur in and around the body 12, this load transmission may be beneficial by permitting more stress to be shared by the newly forming bone. Alternatively, where a stiffer and/or stronger implant 10 is desired, the member 24 may extend through the body 12 such that both the first 26 and second 28 ends are exposed to conduct loads directly between the adjacent bony structures. The tapered portion 30 has a first cross-sectional area adjacent the first end 26 greater than a second cross-sectional area adjacent the second end 28. Also, the side wall of the tapered portion 30 is oblique to the surface of the first end 26 in that the side wall is neither perpendicular nor parallel to the first end 26. The change in area between the first 26 and second 28 ends makes it more difficult to push the member 24 out of the body 12 because of the resultant overlap of a portion of the member 24 over a portion of the body 12 forming a positive engagement between them. Positioning the member 24 with its second end 28 embedded in the body 12 results in further resistance to push-out due to the position of material below the second end 28. The change in area also aids in assembly of the implant 10 by making the member 24 self-aligning as it is inserted into the body 12. The member 24 may optionally be chamfered 25, 27 at one or both ends 26, 28 to relieve stresses in the bone surrounding the member 24 and/or to relieve stresses in the member 24 itself. When the member is inserted into the body 12, stresses may be generated that could lead to flaking of bone or fracturing the body 12 or member 24. Stresses may also be created by subjecting the implant 10 to processes such as cleaning, drying, freezing, rehydrating, or other processes that may differentially affect the body 12 and member 24. Likewise, the body 12 may be chamfered 29 adjacent the member 24 to relieve stresses.

In the illustrative embodiment, the body 12 comprises cancellous bone and the member 24 comprises cortical bone. However, this can be reversed so that the body 12 comprises cortical bone and the member 24 comprises cancellous bone. Cortical bone is denser and stronger than cancellous bone. In the illustrative example, these different mechanical properties result in the member 24 strengthening the implant 10 and increasing its load bearing capacity. It also allows for an intimate fit between the body 12 and member 24 because the cancellous bone surrounding the member 24 may deform to closely fit the shape of the member 24 as the member 24 is inserted into the body 12. In the illustrative embodiment, the body 12 further comprises a cortical face 32 adjacent one end of the body 12. The cortical face extends between the opposing sides 14, 16 and supports the adjacent bony structures. This uni-cortical configuration may be achieved, for example, by harvesting bone from a source having both cortical and cancellous bone and leaving a portion of the cortical bone attached at one end. Alternatively, separate pieces of cancellous and cortical bone may be combined. Similarly, bi-cortical, tri-cortical and other configurations may be employed. In the illustrated example, the cortical face 32 is positioned at one end of the implant 10 and a pair of members 24 are positioned at an opposite end. In a spinal fusion application using the invention, the cortical face 32 and members 24 may be positioned to support the periphery of the endplates of adjacent vertebral bodies while the cancellous bone of the body 12 facilitates growth of bone between the end plates. To further promote fusion, an opening 34 may be provided between the opposing sides 14, 16 of the body to provide a pathway for fusion. The opening 34 may further contain bone growth-promoting material, such as bone paste, cancellous bone, bone chips, bone morphogenic protein (BMP), LIM mineralization protein (LMP), platelet derived growth factors, bone marrow aspirate, stem cells, biologic growth factors, and/or other suitable materials and combinations.

FIG. 3 depicts an alternative arrangement of the members 24 of FIG. 1. By positioning the members 24 opposite one another, their tapered portions 30 can be nested so that a portion 36 of each member 24 overlies a portion of the opposite member 24. This arrangement increases the number of members 24 that can be positioned in a given amount of space 38. Furthermore, these overlying portions 36 may be spaced 40 from one another such that a predetermined amount of load induced subsidence of the members 24 is permitted relative to one another within the body 12. Subsidence may stop when the body material between the overlying portions 36 is sufficiently compressed or when the members 24 come into contact with one another. The members may be positioned so that a portion 36 of each member lies along a line substantially corresponding to a load bearing axis 42 between the opposing bony structures.

In the configuration of FIG. 3, loads are received by a first member which transmits it to the body which in turn transmits it to a second member. In this way, the body acts as a load buffer. For example, loads are transmitted to the members through the first end 26 and distributed from the members to the body through the second end 28 and tapered portion 30. Loads are received from the body by the second end 28 and tapered portion 30 and distributed by the first end 26. This load buffering may be useful, for example, to prevent overloading of adjacent bony structures by permitting some load relieving compression of the body 12 at loads below the compressive strength of the members 24. For example, a controlled amount of subsidence can be designed into a spinal fusion implant to prevent overloading of the vertebral endplates.

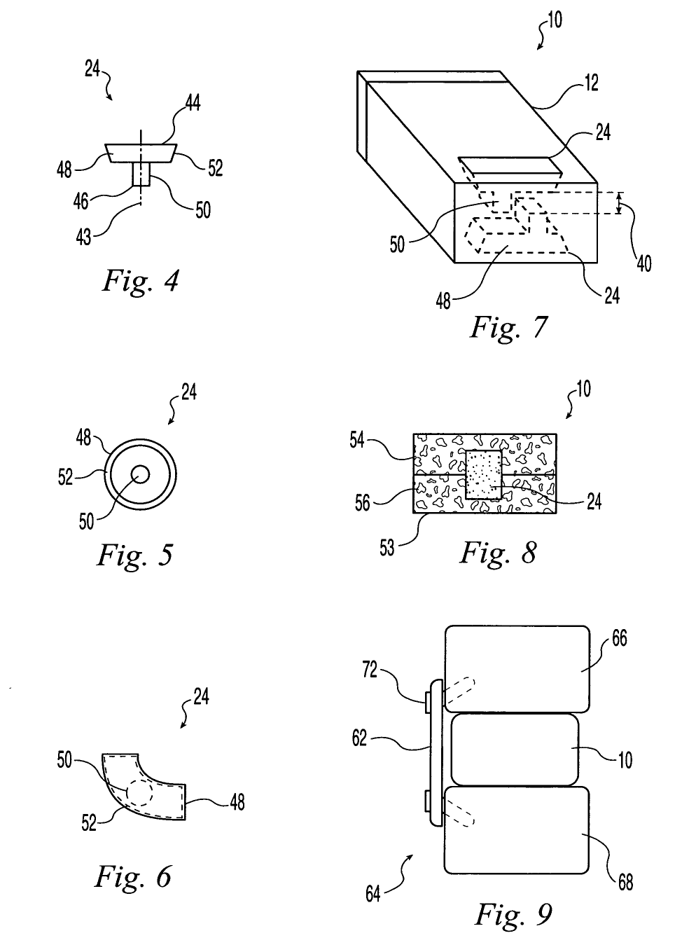

FIGS. 4-7 depict optional members 24 having first and second cross sectional areas along an axis 43. These members 24 include a first end 44, a second end 46, an enlarged head 48 formed adjacent the first end 44, and a shaft 50, or extension, extending from the head 48 to the second end 46. The head has a cross sectional area perpendicular to the axis greater than that of the shaft. The members may further include a tapered portion 52 on the head 48 and/or shaft 50. The illustrative embodiments include a tapered portion 52 on the head 48. FIGS. 4 and 5 depict a member 24 comprising a truncated conical head 48 having a cross sectional area that decreases from the first end 44 toward the second end 46. The shaft 50 comprises a cylinder stepped down in diameter from the smallest head diameter. FIG. 6 depicts a head 48 that describes an arc or approximately 90° and has a cylindrical shaft 50. A tapered portion 52 lies along the perimeter of the head. FIG. 7 depicts an implant 10 having a body 12 similar to that of FIG. 1. The members 24 have a head 48 with a substantially rectangular cross sectional shape and a rectangular shaft 50 extending from the head. The shaft 50 is offset from the center of the head 48. The members 24 are positioned in the body 12 opposite one another with the shaft 50 of one member 24 opposing the head 48 of the other member 24. The members 24 may be spaced such that a predetermined amount 40 of load induced subsidence of the members 24 is permitted relative to one another within the body 12. The members 24 have a first area that receives a load and a second area that transmits the load to the body 12, similar to the configuration of FIG. 3.

FIG. 8 depicts a member 24 embedded in the body 12 such that it is surrounded on all sides by the body. This arrangement eliminates the possibility of the member 24 being dislodged from the body 12. It also permits load induced compression of the body 12 on both sides of the member 24. The implant 10 may be assembled by inserting the member 24 between first 53 and second 54 halves of the body 12.

In all of the above examples, the implant components may be interconnected or joined, such as through mechanical or chemical mechanisms, e.g. pinning, suturing, pressing, incorporating a binding agent, collagen crosslinking, entangling, and other suitable means and combinations thereof.

If the pieces are pinned, holes may be formed in the pieces and rigid pins made of bone, ceramic, metal, polymers, and/or other suitable materials may be pressed into the holes to interconnect the pieces.

If the pieces are sutured together, holes may be formed in the pieces and a flexible, elongate, biocompatible connector may be threaded through the holes to interconnect the pieces. The connector may be a suture and/or elongate pieces of body tissue. Examples of materials for such connectors include pericardium, demineralized bone, fascia, cartilage, tendon, ligament, skin, collagen, elastin, reticulum, intestinal submucosa, metal, resorbable polymer, and nonresorbable polymer, and/or other suitable material.

If a binding agent is used to interconnect the pieces, it may be an adhesive binding agent, a cementitious binding agent, and/or other suitable binding agent. Examples of adhesive binding agents include fibrin glue, cyanoacrylate, epoxy, polymethylmethacrylate, gelatin based adhesives, and other suitable adhesives and combinations thereof. Examples of cementitious binding agents include settable ceramics, calcium carbonate, calcium phosphate, plaster, and other suitable materials and combinations thereof.

If the pieces are interconnected by collagen cross-linking, bone pieces may be partially demineralized to expose collagen fibers which may then be crosslinked by application of heat, pressure, chemicals, and/or other suitable cross-linking means.

Referring to FIG. 9 embodiments of a reinforced fusion implant 10, such as those described above may be utilized in conjunction with a fixation device 62 to form a bone fixation system 64. In such a system 64, the fusion implant 10 is positioned between adjacent bony structures 66, 68 desired to be fused together. The fixation device 62 may include one or more anchor mechanisms 72, such as screws, pins, wires, and/or other mechanisms for attaching it to the adjacent bony structures 66, 68 to limit the relative motion between them. The fixation device 62 may substantially prevent all relative motion, or it may allow a predetermined amount of motion, such as to allow the implant 10 to remain in contact with the adjacent bony structures 66, 68 during the healing and fusion processes. Suitable examples of a fixation device 62 include plates, internal or external rod systems, cable systems, cerclage systems, screws, and other suitable devices and combinations thereof.

Structural members comprising cortical bone may have a predetermined layer thickness and geometry, measured radially from the longitudinal axis of the donor bone, less than a predetermined minimum wall thickness and geometry. For example, the predetermined layer thickness and geometry may be in the range of less than 2 mm thick in one embodiment, less than 1.8 mm thick in another embodiment, less than 1.5 mm thick in yet another embodiment, less than 1.0 mm thick in still another embodiment, and less than 0.5 mm thick in another embodiment. Further, for example, the predetermined minimum wall thickness and geometry may relate to a minimum acceptable thickness or geometry associated with forming an integral or assembled load bearing implant. The predetermined minimum cortical geometry may vary depending on the application. For example, a minimum geometry for use in the cervical spine may be substantially less than a minimum cortical geometry for the lumbar spine. For instance, a predetermined minimum wall thickness or geometry for integral or assembled cortical wedge cervical spine implant, such as may be formed from a fibula, may be 3.0 mm in one embodiment, 2.5 mm in another embodiment, 2.0 mm in yet another embodiment, and 1.8 mm in still another embodiment. On the other hand, a minimum cortical geometry for an integral or assembled lumbar implant may be 4.5 mm in one embodiment, 4.0 mm in another embodiment, and 3.5 mm in another embodiment.

Implants formed from a plurality of bone pieces may have a compressive strength, or load bearing capacity, in the range of 50N to 20,000N. For instance, embodiments may have compressive strength greater than 70N, or greater than 800N, or greater than 100N, or greater than 1200N, or greater than 3000N, or greater than 5000N, or greater than 7000N, or greater than 10,000N, or greater than 12,000N, or greater than 15,000N, or greater than 17,000N. This compressive strength provides load-bearing capability greater than typical cancellous bone and up to that of typical cortical bone.

Although embodiments of implants and methods of making and using them have been described and illustrated in detail, it is to be understood that the same is intended by way of illustration and example only and is not to be taken by way of limitation. Accordingly, variations in and modifications to the implants and methods will be apparent to those of ordinary skill in the art, and the following claims are intended to cover all such modifications and equivalents.

1. A fusion implant for insertion between adjacent bony structures, the implant comprising:
a body having opposing sides for contacting the adjacent bony structures; and
at least one member positioned in the body, the member having a first end and a second end, the member having a tapered portion between the first and second ends.

2. The implant of claim 1 wherein at least one of the first and second ends of the member is chamfered.

3. The implant of claim 1 wherein the member is further connected to the body by incorporating a binding agent.

4. The implant of claim 1 wherein the implant is for insertion between the adjacent bony structures in load bearing arrangement, the body having a load bearing axis extending between its opposing sides, the member being tapered in a direction parallel to the load bearing axis.

5. The implant of claim 4 wherein the member extends from a first opposing side partway toward a second opposing side such that the member is exposed at the first opposing side and stops short of the second opposing side.

6. The implant of claim 5 wherein the tapered portion tapers from the first opposing side toward the second opposing side such that the member has a larger end that is exposed and a smaller end that is contained within the body.

7. The implant of claim 1 wherein the member extends through the body from a first opposing side to a second opposing side.

8. The implant of claim 1 wherein the member is embedded within the body, such that it is surrounded on all sides by the body.

9. The implant of claim 8 wherein the body comprises two halves that fit together around the member.

10. The implant of claim 1 wherein the body and the member comprise materials having different mechanical properties.

11. The implant of claim 10 wherein the body comprises a material selected from the group consisting of cancellous bone, cortical bone, uni-cortical bone, bi-cortical bone, tri-cortical bone, demineralized bone, partially demineralized bone, metal, polymer, resorbable polymer, ceramic, and bioglass.

12. The implant of claim 10 wherein the member comprises a material selected from the group consisting of cancellous bone, cortical bone, uni-cortical bone, bi-cortical bone, tri-cortical bone, demineralized bone, partially demineralized bone, metal, polymer, resorbable polymer, ceramic, and bioglass.

13. The implant of claim 10 wherein the body has a portion surrounding the member and one of the portion and member comprises cancellous bone and the other of the portion and member comprises cortical bone.

14. The implant of claim 13 wherein the portion of the body surrounding the member comprises cancellous bone and the member comprises cortical bone.

15. The implant of claim 14 wherein the body further comprises at least one cortical face adjacent the cancellous bone, the cortical face extending between the opposing sides of the body.

16. The implant of claim 10 wherein the body has a portion surrounding the member, the portion surrounding the member being relatively softer than the member, the member being fit into the body such that the portion surrounding the member is deformed to fit closely around the member.

17. The implant of claim 1 wherein the member has a first end having a first cross sectional area and a second end having a second cross sectional area, the first cross sectional area being larger than the second cross sectional area.

18. The implant of claim 17 wherein the member comprises an enlarged head adjacent the first end and a shaft extending from the head to the second end.

19. The implant of claim 1 wherein the opposing sides comprise first and second opposing sides and at least one member extends from each of the first and second opposing sides partway toward the other opposing side, a portion of the at least one member extending from each side overlying a portion of the at least one member extending from the opposite side, the overlying portions being spaced from one another such that a predetermined amount of load induced subsidence of the members is permitted relative to each other within the body.

20. The implant of claim 1 wherein at least one member extends from each of the opposing sides partway toward the other opposing side, each member having a first area for receiving a load from the adjacent bony structures and a second area for transmitting the load to the body.

21. The implant of claim 1 further comprising a fixation device attached to the adjacent bony structures to limit the relative motion between them.

22. The implant of claim 21 wherein the fixation device substantially prevents all relative motion between the adjacent bony structures.

23. The implant of claim 21 wherein the fixation device allows a predetermined amount of relative motion between the adjacent bony structures during the fusion process.

24. The implant of claim 21 wherein the fixation device is selected from the group consisting of plates, internal rod systems, external rod systems, cable systems, cerclage systems, screws, and combinations thereof.

25. A fusion implant for insertion between adjacent bony structures in load bearing arrangement, the implant comprising:
a body comprising bone and having opposing sides for contacting the adjacent bony structures; and
a reinforcing member comprising bone positioned in the body such that the load carrying capacity of the implant is increased, the member having a first end and a second end, the member having a tapered portion between the first and second ends.

26. A fusion implant for insertion between adjacent bony structures in load bearing arrangement, the implant comprising:
a body having opposing sides for contacting the adjacent bony structures; and
a structural member positioned in the body such that the load carrying capacity of the implant is increased, the member having a first end and a second end, the member extending only partway through the body.

27. The implant of claim 26 wherein the member is embedded in the body such that it is surrounded on all sides by the body.

28. A fusion implant for insertion between adjacent bony structures in load bearing arrangement, the implant comprising:
a body having first and second opposing sides for contacting the adjacent bony structures; and
a member positioned in the body, the member having a first end having a first cross sectional area adjacent the first opposing side and a second end having a second cross sectional area spaced toward the second opposing side, the first cross sectional area being larger than the second cross sectional area.

29. The implant of claim 28 wherein the member comprises an enlarged head formed adjacent the first end and a shaft extending from the head toward the second end.

30. A fusion implant for insertion between adjacent bony structures in load bearing arrangement, the implant comprising:
a body having first and second opposing sides for contacting the adjacent bony structures; and
at least one member positioned in the body and extending from each of the first and second opposing sides partway toward the other opposing side, a portion of the at least one member extending from each side overlying a portion of the at least one member extending from the opposite side, the overlying portions being spaced from one another such that a predetermined amount of load induced subsidence of the members is permitted relative to each other within the body.

31. A fusion implant for insertion between adjacent bony structures in load bearing arrangement, the implant comprising:
a body having first and second opposing sides for contacting the adjacent bony structures; and
a member positionable to extend within the body from at least one of the first and second opposing sides, the member having a first surface that receives a load from one of the bony structures and a second surface, oblique to the first surface, that transmits the load to the body.

32. The implant of claim 31, wherein a size of the second surface is greater than a size of the first surface.

33. The implant of claim 31, wherein the member includes a tapered portion.

34. The implant of claim 31, wherein the body further comprises a first load bearing capability, and wherein the member further comprises a second load bearing capability greater than the first load bearing capability.

35. A fusion implant for insertion between adjacent bony structures in load bearing arrangement, the implant comprising:
a body having first and second opposing sides for contacting the adjacent bony structures; and
a member positionable to extend within the body from at least one of the first and second opposing sides, the member having a first portion with a first cross-sectional area that receives a load from one of the bony structures and a second portion with a second cross-sectional area that transmits the load to the body.

36. The implant of claim 35 wherein the first cross-sectional area is larger than the second cross sectional area, the first portion being adjacent one of the first and second opposing sides and the second portion being spaced from the first portion into the body.

37. A fusion implant for insertion between adjacent bony structures in load bearing arrangement, the implant comprising:
a body having first and second opposing sides for contacting the adjacent bony structures;
a first member positionable to extend within the body from one of the first and second opposing sides, the first member comprising a first body having a first end and a second end, wherein the first end is positionable adjacent one of the opposing bony structures, and wherein the first body has a tapered portion between the first and second ends;
a second member positionable to extend within the body from one of the first and second opposing sides, the second member comprising a second body having a third end and a fourth end, wherein the third end is positionable adjacent one of the opposing bony structures, and wherein the second body has a tapered portion between the first and second ends; and
wherein at least a portion of the second member and at least a portion of the first member each lie along a line substantially corresponding to a load bearing axis between the opposing bony structures.

38. The implant of claim 37, wherein the body further comprises a first load bearing capability, and wherein the implant comprises a second load bearing capability greater than the first load bearing capability.

39. A fusion implant for insertion between adjacent bony structures in load bearing arrangement, the implant comprising:
a body having first and second opposing sides for contacting the adjacent bony structures;
a first member positionable to extend within the body from one of the first and second opposing sides;
a second member positionable to extend within the body from one of the first and second opposing sides opposite the first member; and
wherein at least a portion of the first member and at least a portion of the second member each lie along a line substantially corresponding to a load bearing axis between the opposing bony structures, the body having a first area that receives load from the first member and a second area that transmits load to the second member.

40. The implant of claim 39, wherein the first and second members are spaced from one another along the load bearing axis such that a predetermined amount of load induced subsidence of the members is permitted relative to each other within the body.

41. A system for use in fusing adjacent bony structures, comprising:
a body having first and second opposing sides for contacting the adjacent bony structures;
a member positionable to extend within the body from at least one of the first and second opposing sides, the member having a first surface that receives a load from one of the bony structures and a second surface, oblique to the first surface, that transmits the load to the body; and
a fixation device attachable to the adjacent bony structures and having a structure to limit relative motion between the adjacent bony structures.

42. A system for use in fusing adjacent bony structures, comprising:
a body having first and second opposing sides for contacting the adjacent bony structures;
a member positionable to extend within the body from at least one of the first and second opposing sides, the member having a first portion with a first cross sectional area that receives a load from one of the bony structures and a second portion with a second cross sectional area that transmits the load to the body; and
a fixation device attachable to the adjacent bony structures and having a structure to limit relative motion between the adjacent bony structures.

43. A method of treating adjacent bony structures comprising:
providing a fusion implant having a body having opposing sides for contacting the adjacent bony structures and a member positioned in the body, the member having a first end and a second end, the member having a tapered portion between the first and second ends; and
positioning the implant between the adjacent bony structures in load bearing arrangement.

44. The method of claim 43 wherein the implant is positioned such that the member receives a portion of the load from the adjacent bony structures and transmits a portion of the load to the body.

45. A method of treating adjacent bony structures comprising:
providing a fusion implant having a body having opposing sides for contacting the adjacent bony structures and a member positioned in the body, the member having a first end and a second end, the member extending only partway through the body; and
positioning the implant between the adjacent bony structures in load bearing arrangement.

46. A method of treating adjacent bony structures comprising:
providing a fusion implant having a body having first and second opposing sides for contacting the adjacent bony structures and at least one member positioned in the body and extending from each of the first and second opposing sides partway toward the other opposing side, a portion of the at least one member extending from each side overlying a portion of the at least one member extending from the opposite side, the overlying portions being spaced from one another such that a predetermined amount of load induced subsidence of the members is permitted relative to each other within the body; and
positioning the implant between the adjacent bony structures in load bearing arrangement.

47. A method of making a fusion implant for insertion between adjacent bony structures in load bearing arrangement, the method comprising:
forming a body; and
positioning a member in the body, the member having a first end and a second end, the member having a tapered portion between the first and second ends.

48. A method of making a fusion implant for insertion between adjacent bony structures in load bearing arrangement, the method comprising:
forming a body having opposing sides for contacting the adjacent bony structures; and
positioning a member in the body, the member having a first end and a second end, the member extending only partway through the body.

49. A method of making a fusion implant for insertion between adjacent bony structures in load bearing arrangement, the method comprising:
forming a body having first and second opposing sides for contacting the adjacent bony structures; and
positioning at least one member in the body extending from each of the first and second opposing sides partway toward the other opposing side, a portion of the at least one member extending from each side overlying a portion of the at least one member extending from the opposite side, the overlying portions being spaced from one another such that a predetermined amount of load induced subsidence of the members is permitted relative to each other within the body.
